(19) United States
(12) Patent Application Publication
(10) Pub. No.: US 2011/0215002 A1
(43) Pub. Date: Sep. 8, 2011

(54) Sensing device and related methods

(76) Inventor: William Emerson Martinez, Santa Barbara, CA (US)

(21) Appl. No.: 12/931,818

(22) Filed: Feb. 11, 2011

Related U.S. Application Data
(60) Provisional application No. 61/338,214, filed on Feb. 16, 2010.

(51) Int. Cl. G01N 33/50; G01N 27/30; G01N 27/327; G01N 27/26; B82Y 5/00; B82Y 99/00

(52) U.S. Cl. 205/777.5; 204/435; 204/403.01; 205/790; 205/792; 977/750; 977/953; 977/958

(57) ABSTRACT

The present invention is generally directed to devices and methods for sensing a variety of biologically-related substances. In a device aspect, the present invention is directed to a multilayer device for sensing metal ions, biological molecules, or whole cells. The device comprises: a) one or more cavities that provide for the introduction of a sample to be analyzed and one or more channels that provide for exit of the sample, or one or more channels that provide for the introduction and exit of the sample; b) one or more single-walled carbon nanotubes presented to the one or more cavities or one or more channels; c) a plurality of electrodes electrically connected to the one or more single-walled carbon nanotubes; and, a reference gate electrode presented to the one or more cavities or one or more channels. In a method aspect, the present invention is directed to a method for sensing species such as a metal, biological cells, and one or more biological molecules using the device.

This application claims the benefit of U.S. Provisional Application No. 61/338,214 filed Feb. 16, 2010, which is incorporated-by-reference for all purposes.

### FIELD OF THE INVENTION

The present invention is generally directed to devices and methods for sensing a variety of biologically-related substances.

### BACKGROUND OF THE INVENTION

There is a growing need for reliable and low-cost early cancer screening technologies that could enable physicians to detect cancers at early stages when the diseases are most treatable and treatments offer better outcomes for patients. The global market for in-vitro diagnostics (IVD) systems for cancer reached US$ 3.39 billion in 2008 and continues to grow. This increasing demand is the result of the growing costs associated with treating and battling cancer, which in 2008 reached $228 billion in the US alone, where 562,340 people died and 1,479,350 new cancer cases were diagnosed in 2009. (ACS, Facts & Figures 2009).

In its 2007 report, the National Institute of Health (NIH) provided estimates for the growing costs and expenditures related to battling cancer: direct medical costs and health expenditures ($89.0 billion); indirect morbidity costs due to lost productivity and illness ($18.2 billion); and, indirect mortality costs due to productivity loss and premature death ($112.0 billion).

One barrier to reducing the staggering number of cancer-related deaths and resulting health care costs is the lack of accurate, reliable and low cost early detection methods. The emerging field of precise molecular diagnostics provides windows of opportunity for the early detection of cancers, among other diseases, because it can enable the detection of molecular biomarkers and biological analytes at very small concentrations. Emerging molecular diagnostic technologies provide opportunities for early cancer detection, as they can enable the detection of minute quantities of biomarker arrays. Current methods, however, are costly and time intensive: they require extensive sample preparation, complex hardware, sophisticated instrumentation and hours to days of analysis.

### SUMMARY OF THE INVENTION

The present invention is generally directed to devices and methods for sensing a variety of biologically-related substances.

The present invention addresses the need for rapid, accurate, reliable and low cost detection methods. It can detect analytes at very low concentrations in gases and fluids, including the sensing of a variety of biologically-related substances, thereby facilitating the detection and screening of diseases. The present invention can also be used in the detection of biological species for national security. Other applications include the detection of metals, pollutants, biologically-related species in ground water, sea water and other water sources (environmental monitoring and remediation).

In a device aspect, the present invention is directed to a multilayer device for sensing metal ions, biological molecules, or whole cells. The device comprises: a) one or more cavities that provide for the introduction of a sample to be analyzed and one or more channels that provide for exit of the sample, or one or more channels that provide for the introduction and exit of the sample; b) one or more single-walled carbon nanotubes presented to the one or more cavities or one or more channels; c) a plurality of electrodes electrically connected to the one or more single-walled carbon nanotubes; and, a reference gate electrode presented to the one or more cavities or one or more channels.

In a method aspect, the present invention is directed to a method for sensing species such as a metal, biological cells, and one or more biological molecules. The method comprises the steps of: a) introducing a solution of high affinity and selective binding elements into a device discussed above in the Summary of Invention Section, wherein the high affinity and selective binding elements add functionality to the one or more single-walled carbon nanotubes by binding species of interest to the surface of the nanotubes; b) introducing a buffer-electrolyte solution into one or more cavities, or one or more channels of the device, thereby allowing activation of nanotube-field effect transistors in the device for calibration and for setting a baseline current or voltage reference state; c) introducing a sample in solution with a buffer-electrolyte solution into the one or more cavities, or one or more channels of the device and determining any changes in the current or voltage state of the nanotube-field effect transistors relative to their baseline state. The changes are correlated with the binding of one or more species of interest in the sample.

### BRIEF DESCRIPTION OF THE FIGURES

FIG. 1 shows a three-dimensional perspective sketch for the main components of one of the layers of a sensor according to the present invention.

FIGS. 2-8 show side view cross-sections of multiple different embodiments according to the present invention.

FIG. 9 shows a three-dimensional perspective sketch for the main components of one of the layers of a sensor according to the present invention.

FIGS. 10-19 show side view cross-sections of multiple different embodiments according to the present invention.

### DETAILED DESCRIPTION OF THE INVENTION

### Definitions

“Cavity” refers to an unfilled space within a mass or substrate.

“Channel” refers to an enclosed passage between substrates or within a substrate.

“Microchannel” refers to an enclosed passage with micro-scale dimensions between substrates.

“Electrode” refers to a conductor used to establish electrical contact with a nonmetallic part of a circuit.

### DETAILED DESCRIPTION

The present invention may be used to detect a variety of substances, including clusters of atoms (e.g., Hg, Au, and Pb), specific ions, molecules, biologically-related substances (e.g., molecules and macromolecules, such as proteins, RNA and DNA), and whole biological cells. The sensor comprises carbon nanotubes, which interact with atoms and molecules in their surroundings. The affinity of the nanotubes for specific target analytes and species is enhanced by the binding of high affinity and selective elements such as aptamers, peptides, enzymes, antibodies, antibody fragments (e.g. minibodies, diabodies, cys-diabodies, Fab fragments and F(ab′)2 fragments), or a combination thereof onto the surface of the nanotubes. These high affinity and selective elements serve as links between nanotubes and analytes of interest such that their interaction can be enhanced, detected and quantified at very low analyte concentrations (e.g., nano-, pico-, and femto-molar concentrations).

The sensor has the capability to separate and decouple microfluidic control and circulation from ionic, electrochemical, and/or electrostatic detection. The microfluidics, for example, may be controlled from one side of the device; and the electronic and electrical input/outputs for detection can be controlled from the opposite side of the device.

The sensor may be used in a variety of applications. These applications include, but are not limited to, the following: disease detection, including early disease detection and screening; diagnostics; and, monitoring of analytes for therapeutic intervention. Other potential applications include analyte detection for water quality control, environmental monitoring of underground water resources and detection of underground contaminants, environmental monitoring of water reservoirs and sea water, monitoring of potable water for protection against biological and biochemical terrorism, and strategic monitoring of water resources for national security.

The present invention can be classified onto two main kinds of embodiments: open cavity embodiments and enclosed microchannel embodiments. First, the open cavity embodiments are described starting with the building-block components and elements that are critical to the invention. Next, the enclosed microchannel embodiments are described including the building-block components and elements that are critical to the present invention. Subsequently, utility and functional advantages of the present invention are described. This section ends with a description of the method of detection and analysis that is attainable with the present invention.

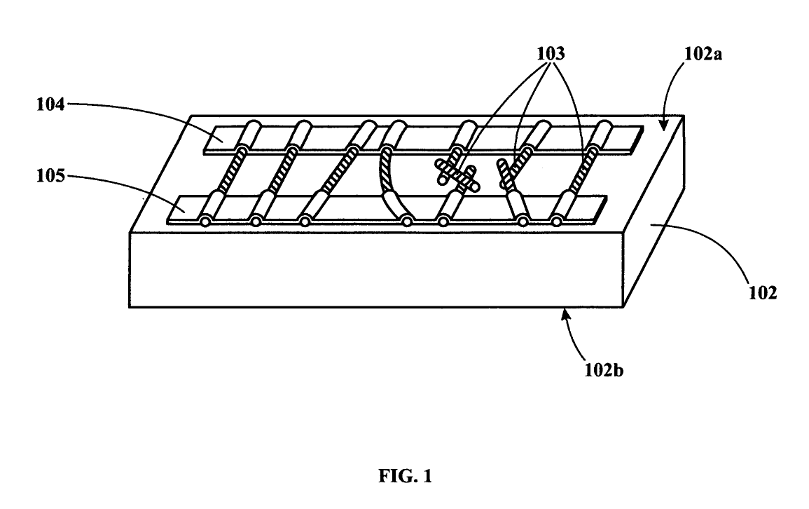

Open-Cavity Embodiments: FIGS. 1-8

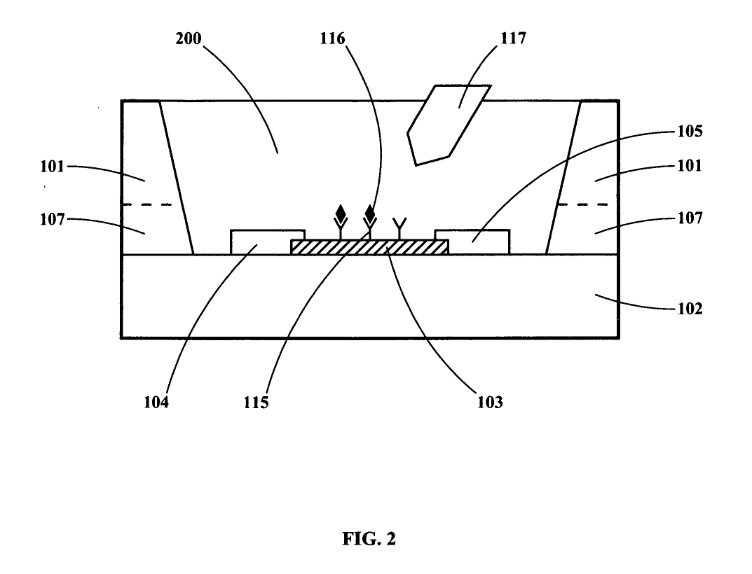

Elements of the present invention are described in FIG. 1, which shows an array of single-walled carbon nanotubes (SWCNT) 103 on the front surface 102a of a layer, so-called substrate 102. The nanotubes 103 are electrically connected in parallel, in series, or a combination thereof at one end by a source electrode 104 and at the second end by a drain electrode 105. The second component of the present invention is substrate 101. One embodiment of the present invention is described in reference to FIG. 2, where substrate 101 comprises a through-substrate cavity (TSC) 200. FIG. 2 shows a lateral cross-section diagram of substrates 101 and 102. In this embodiment, the biosensor is comprised of two substrates that come together in “face-to-face” fashion. Substrate 101 comprises the TSC 200 and microchannels 107 that allow for the introduction and exit of a sample during analysis. Substrate 102 comprises nanotube 103, source electrode 104, and drain electrode 105. An external gate electrode probe 117 is inserted into the sensing cavity 200, also referred to as TSC, where the sample is introduced. During detection and analysis, target analytes 116 bind to high affinity species 115 on the surface of the nanotube 103.

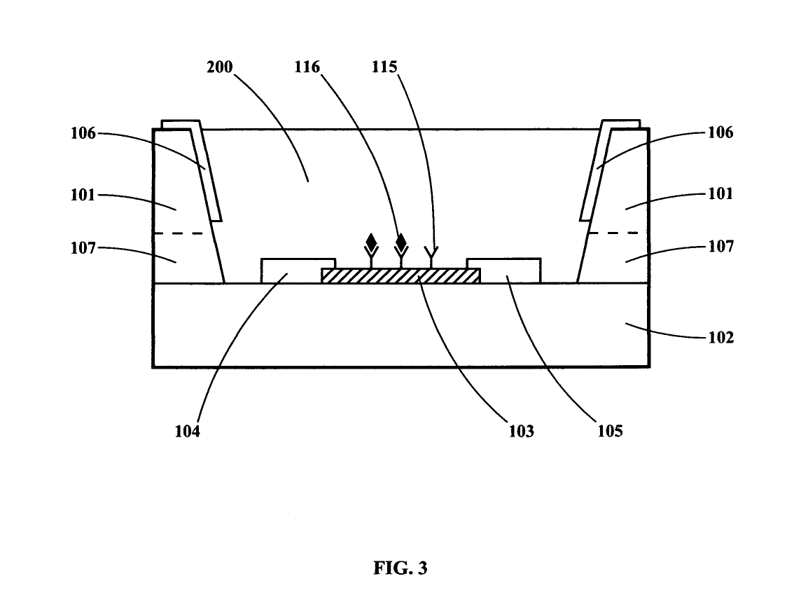

A slightly different embodiment of the present invention is described in reference to FIG. 3, which also shows a lateral cross-section diagram of substrates 101 and 102. In this embodiment, the biosensor is also comprised of two substrates that come together in “face-to-face” fashion. Substrate 101 comprises the sensing TSC 200 and microchannels 107 that allow for the introduction and exit of a sample during analysis. No external gate electrode probe is used. Instead, a gate electrode 106 runs along the sidewall of the sensing TSC 200 and extends to the top surface of substrate 101. Substrate 102 comprises nanotube 103, source electrode 104 and drain electrode 105. As displayed, target analytes 116 bind to high affinity species 115 on the surface of nanotube 103 during sensing and analysis.

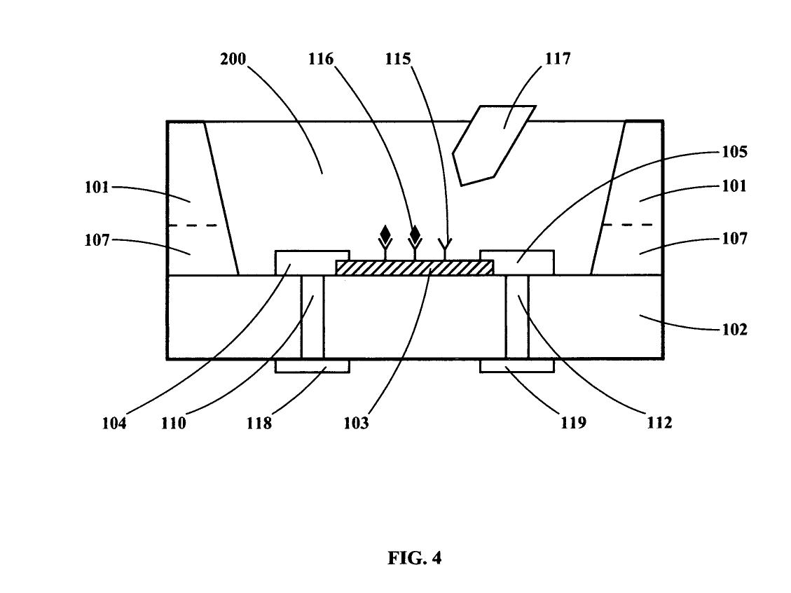

Another embodiment of the present invention is described in reference to FIG. 4, which also shows a lateral cross-section diagram of substrates 101 and 102. In this embodiment, the biosensor is comprised of two substrates that come together in “face-to-face” fashion. Substrate 101 comprises the sensing TSC 200 and microchannels 107 that allow for the introduction and exit of a sample during analysis. Substrate 102 comprises nanotube 103, source electrode 104, drain electrode 105. and through-substrate vias (TSV) 110 and 112, which are connected to the source electrode 104 and drain electrode 105, respectively. Metal traces 118 and 119 on the back surface of substrate 102 are connected to TSVs 110 and 112, respectively. These metal traces 118 and 119 are points of electrical connection to external power supply systems and/or devices. An external gate electrode probe 117 is inserted into the sensing cavity 200 for analysis and detection when target analytes 116 bind to high affinity species 115 on the surface of nanotube 103.

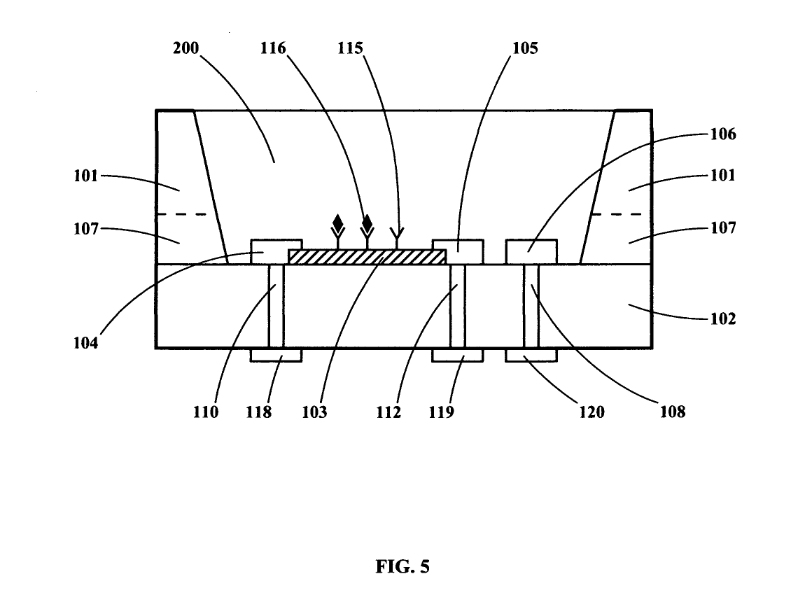

Another embodiment of the present invention is described in reference to FIG. 5, which also shows a lateral cross-section diagram. Similarly, the biosensor is comprised of two substrates 101 and 102 that come together in “face-to-face” fashion. Substrate 101 comprises the sensing TSC 200 and microchannels 107 that allow for the introduction and exit of a sample during analysis. Substrate 102 comprises nanotube 103, source electrode 104, drain electrode 105, and through-substrate vias (TSV) 110 and 112, which are connected to the source 104 and drain electrode 105 respectively. Metal traces 118 and 119 on the back surface of substrate 102 are connected to TSVs 110 and 112, respectively. In this embodiment there is no external gate probe, but there is a gate electrode 106 on the front surface of substrate 102. Gate electrode 106 is connected to TSV 108, which is connected to metal trace 120 on the back surface of substrate 102. Therefore, metal traces 118, 119, and 120 on the back surface of substrate 102 are electrically connected to electrodes 104, 105, and 106, respectively. Target analytes 116 bind to high affinity species 115 on the surface of nanotube 103 during sensing and analysis.

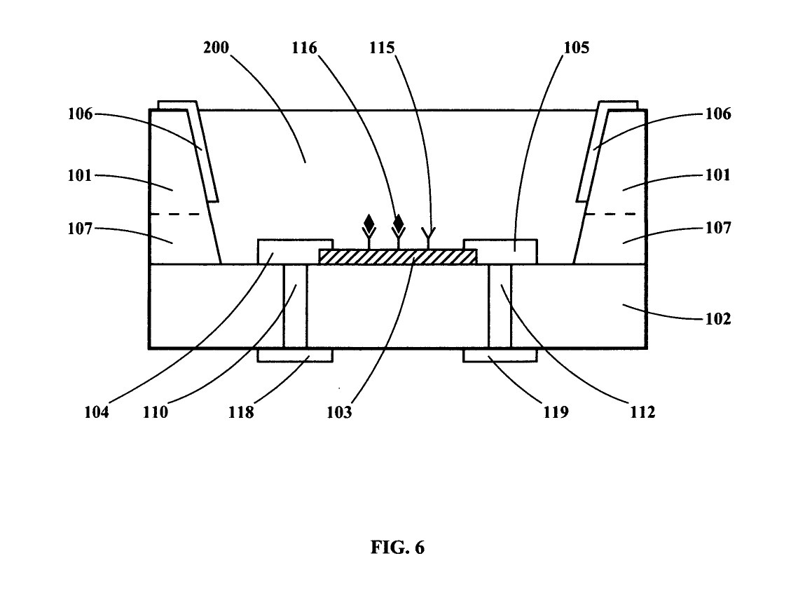

An alternative embodiment of the present invention is described in reference to FIG. 6. Instead of utilizing an external gate probe, this embodiment comprises a gate electrode 106 that runs along the sidewall of sensing TSC 200 and extends to the top surface of substrate 101. Substrate 101 comprises the sensing TSC 200 and microchannels 107 that allow for the introduction and exit of a sample during analysis. Substrate 102 comprises nanotube 103, source electrode 104 and drain electrode 105. Source electrode 104 is electrically connected to metal trace 118 via TSV 110. Similarly, drain electrode 105 is electrically connected to metal trace 119 via TSV 112. During sensing and analysis, target analytes 116 bind to high affinity species 115 on the surface of nanotube 103.

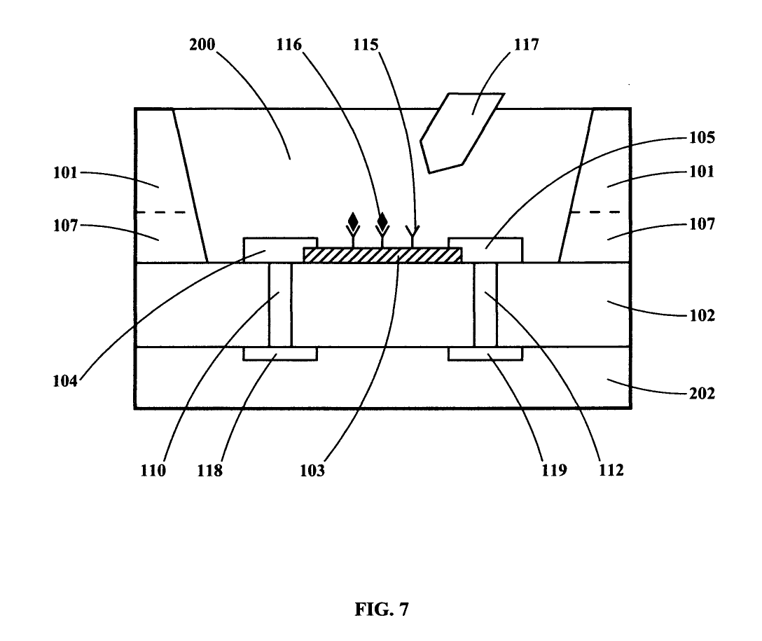

FIG. 7 displays an embodiment of the present invention that is similar to the embodiment described in FIG. 4. In addition to comprising all the different elements described in FIG. 4, this embodiment further comprises an integrated circuit 202, which is attached to the back surface of substrate 102. The connection of the integrated circuit 202 to the metal traces 118, 119, and 120 enables additional miniaturization of the biosensor since electrical inputs and outputs can be programmed, controlled, and recorded by the integrated circuit 202 during sensing and analysis.

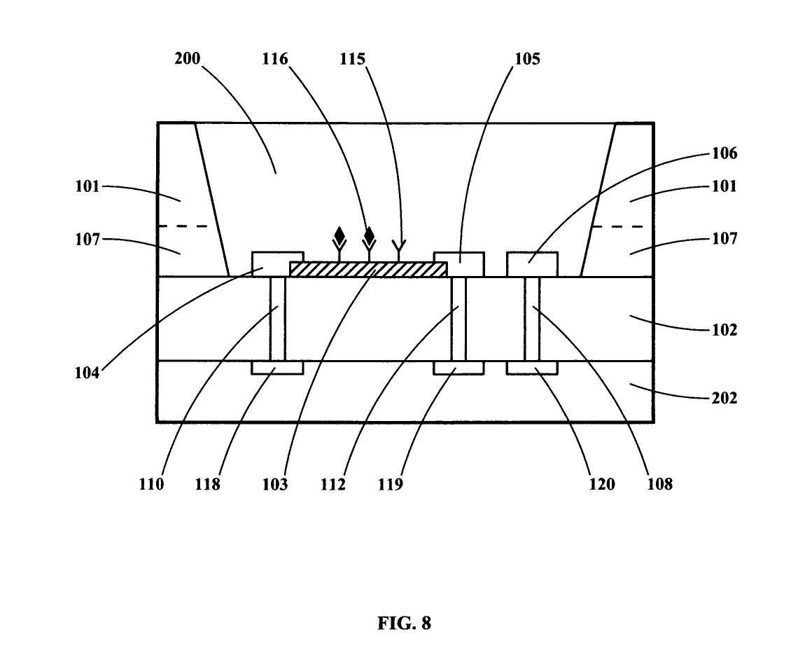

FIG. 8 displays an embodiment of the present invention that is similar to the embodiment described in FIG. 5, where a gate electrode 106 is located on the front surface of substrate 102, and said gate electrode 106 is electrically connected to metal trace 120 via TSV 108. In addition to comprising all the different elements described in FIG. 5, this embodiment further comprises an integrated circuit 202, which is attached to the back surface of substrate 102. The connection of the integrated circuit 202 to the metal traces 118, 119, and 120 enables additional miniaturization of the biosensor because electrical inputs and outputs can be programmed, controlled, and recorded by the integrated circuit 202 during sensing and analysis.

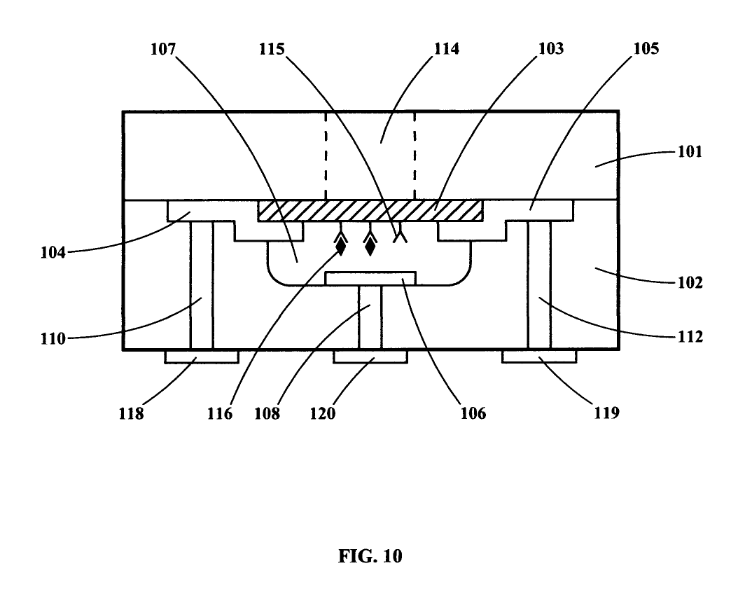

Enclosed Microchannel Embodiments: FIGS. 10-19

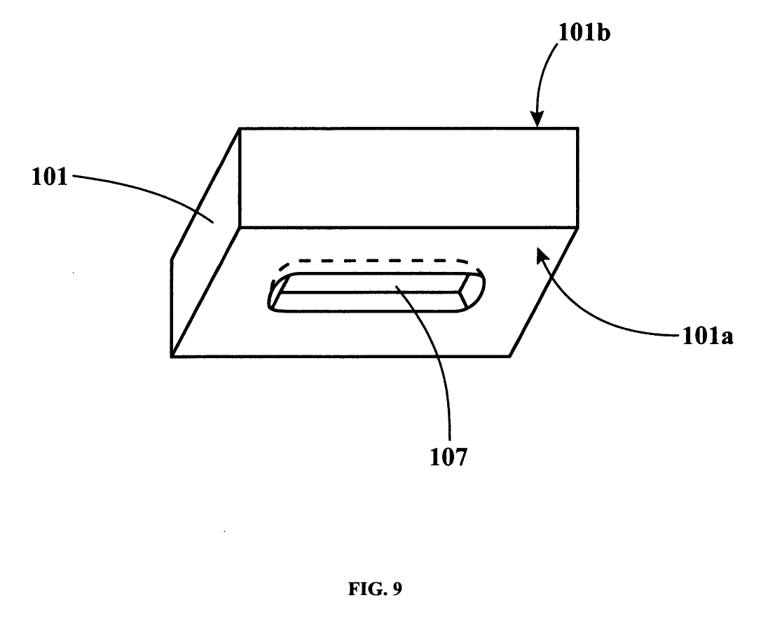

Elements for this family of embodiments are described in FIG. 1, which displays an array of single-walled carbon nanotubes (SWCNT) 103, a source electrode 104, and a drain electrode 105 on a layer, so-called substrate 102. Another critical element is described with reference to FIG. 9, which shows a microchannel 107 on the bottom surface 101a of a layer, so-called substrate 101. In reference to FIG. 1, the nanotubes 103 are electrically connected in parallel, in series, or a combination thereof at one end by a source electrode 104 and at the second end by a drain electrode 105. In reference to FIG. 9, substrate 101 comprises one or a plurality of horizontal microchannels 107, which are connected to a plurality of vertical channels. The embodiments described in this section have at least two substrates 101 and 102, which come together in “face-to-face” fashion. Substrate 101 has a front surface 101a and a back surface 101b (FIG. 9), and substrate 102 has a front surface 102a and a back surface 102b (FIG. 1). The enclosed channel embodiments are subdivided into two groups: Embodiments that have nanotubes 103 on surface 101a and microchannels 107 on surface 102a, and embodiments that have nanotubes on surface 102a and microchannels 107 on surface 101a are also envisioned.

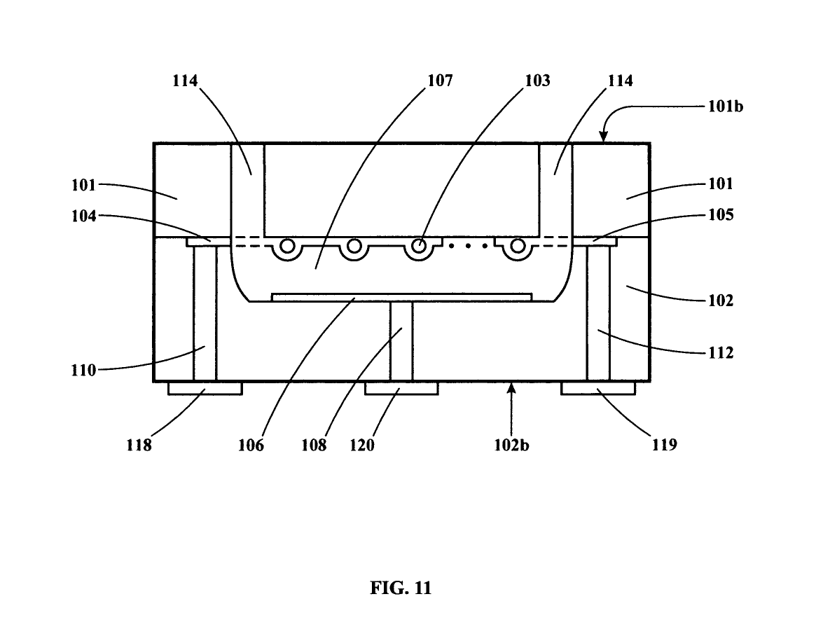

One embodiment of the present invention is described in reference to FIG. 10, which shows a side view cross-section diagram of substrates 101 and 102. Substrate 101 comprises vertical channels 114, nanotubes 103 connected to source electrode 104 and drain electrode 105. Vertical channels 114 allow for the introduction and exit of a sample to microchannel 107 on substrate 102 during sample analysis. Substrate 102 comprises microchannel 107, gate electrode 106, TSV 108, and metal trace 120. Source electrode 104 is connected to metal trace 118 via TSV 110. Similarly, drain electrode 105 is connected to metal trace 119 via TSV 112. Target analytes 116 bind to high affinity species 115 on the surface of nanotube 103 during sample sensing and analysis. A different side view cross-section of this embodiment is displayed in FIG. 11.

In reference to FIG. 11, an array of nanotubes 103 are present on the front surface of substrate 101; microchannels 107 are etched or mechanically formed on the front surface of substrate 102. Electrically conductive through-layer means, which are also referred to as through-substrate vias (TSVs), are included in substrate 102. These through-layer conductive vias are the shortest path of electrical connection between the front side and the back side of substrate 102. Gate electrode 106 runs along microchannel 107 on the front surface of substrate 102. This integrates source electrode 104, nanotubes 103, gate electrode 106, and drain electrode 105 into one or a plurality of functional nanotube field effect transistors (NT-FETs), which can be operated and controlled from the back surface of substrate 102 when using an external power supply and an integrated circuit/system.

Vertical channels 114 connect both sides of substrate 101 such that a fluid or gas sample can flow from back side 101b into sensing microchannel 107, then through a second set of vertical channels 114 back to surface 101b to exit the device. In this embodiment, microfluidic control is conducted from surface 101b. The electronic current/voltage (“I/V”) characteristics are controlled from back side 102b using an external integrated circuit and power supply.

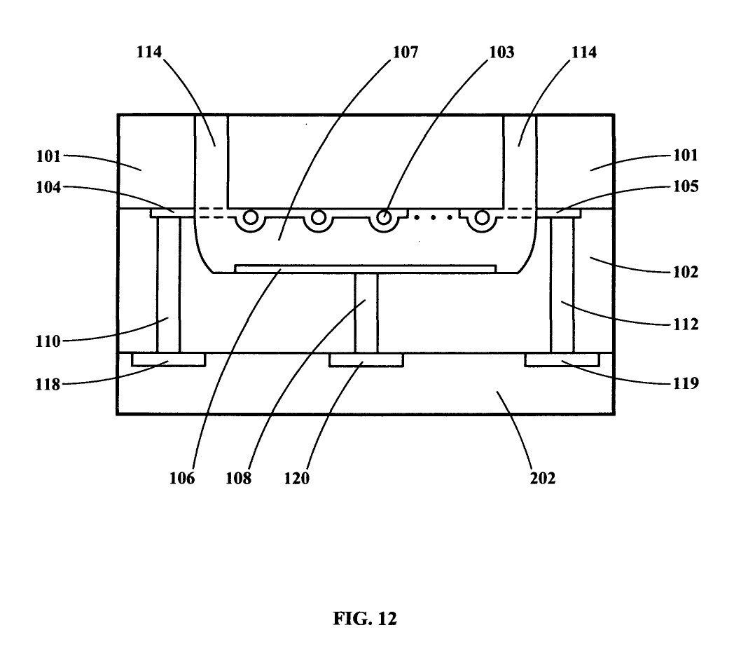

FIG. 12 displays an embodiment of the present invention that is similar to the embodiment described in FIG. 11. In addition to comprising all the different elements described for the previous embodiment in FIG. 11, this embodiment comprises an integrated circuit 202, which is attached to the back surface of substrate 102. The integrated circuit 202 is connected to the metal traces 118, 119, and 120. This enables additional miniaturization of the biosensor since electrical inputs and outputs can be programmed, controlled, and recorded by the integrated circuit 202.

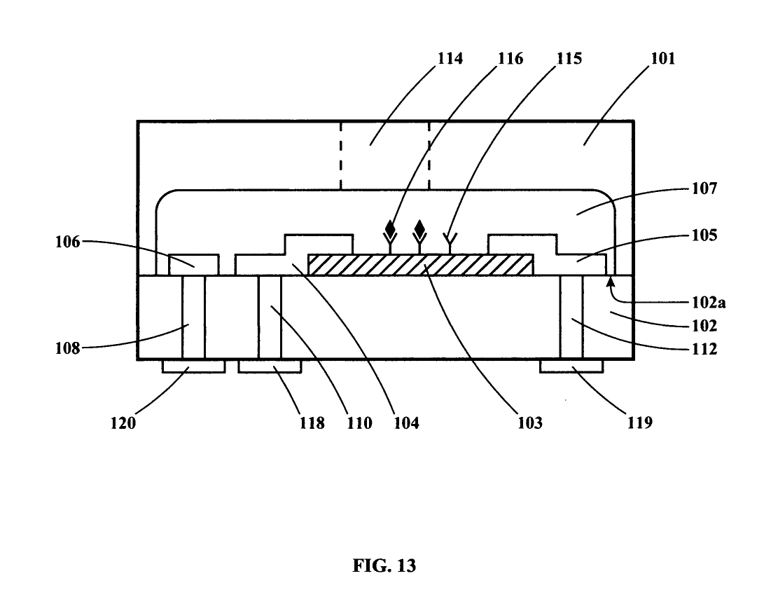

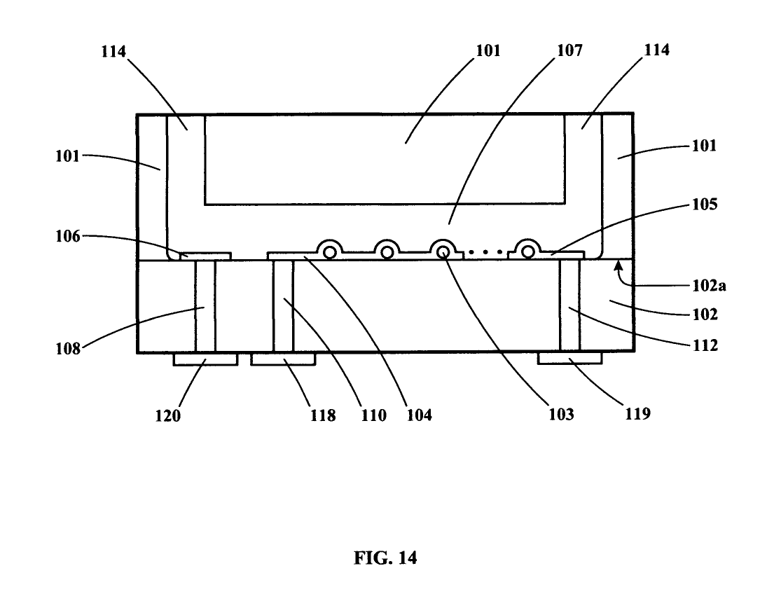

Having the nanotubes on surface 102a (FIG. 1) and microchannel 107 on surface 101a (FIG. 9) gives rise to multiple embodiments. One embodiment of the present invention with this characteristic is described in reference to FIG. 13, which shows a lateral cross-section diagram. Similarly, the biosensor is comprised of two substrates 101 and 102 that come together in “face-to-face” fashion. Substrate 102 comprises nanotubes 103, source electrode 104, drain electrode 105, and gate electrode 106 on surface 102a. The electrodes 104, 105, and 106, are connected to metal traces 118, 119, and 120 via TSVs 110, 112, and 108, respectively. Substrate 101 comprises microchannels 107 and vertical channels 114 for the introduction and exit of a sample during detection and analysis. Target analytes 116 bind to high affinity species 115 onto the surface of nanotube 103 during sensing and analysis. This embodiment is also displayed in FIG. 14, but the view corresponds to an orthogonal side view cross-section where an array of nanotubes 103 are visible on surface 102a. All the elements described in FIG. 13 are also present in this figure.

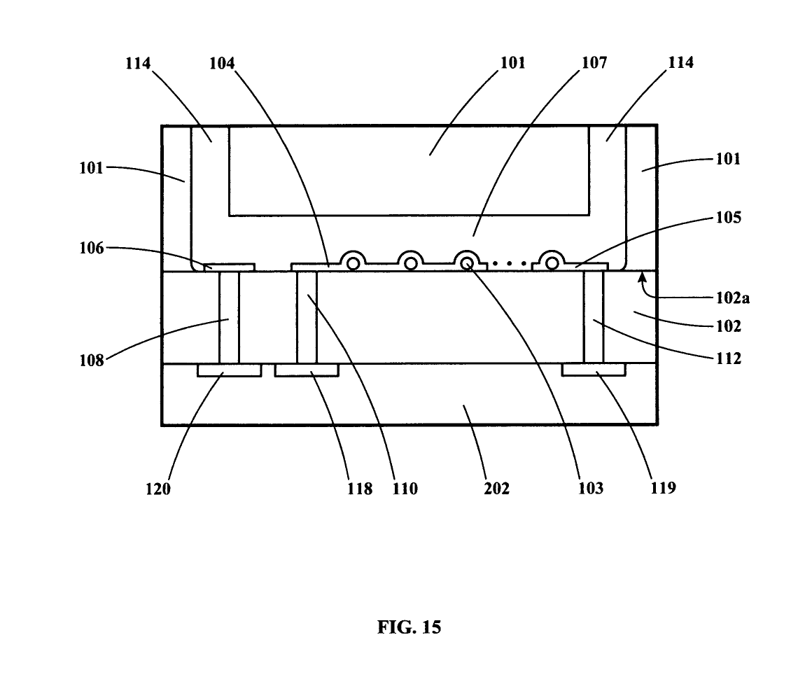

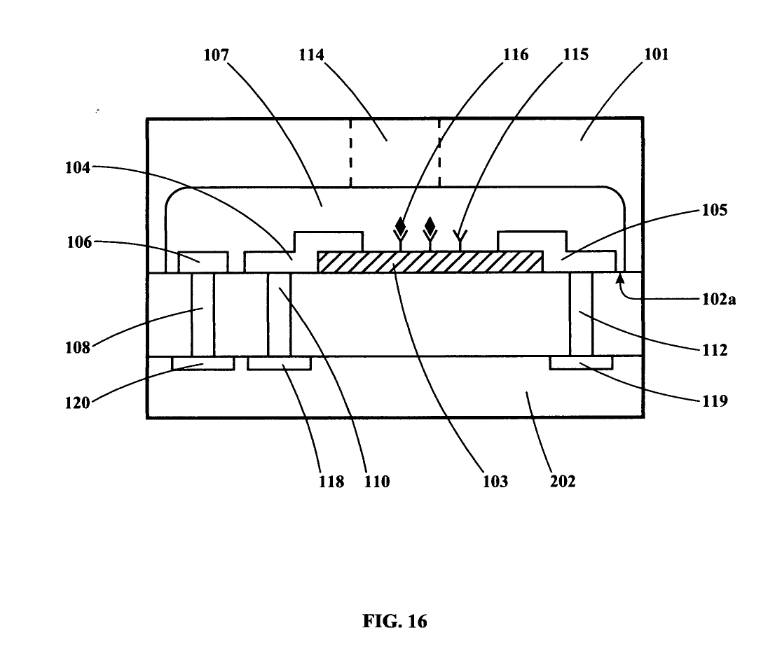

A similar embodiment is displayed with reference to FIG. 15. In addition to all the elements described in FIG. 13 and FIG. 14, this embodiment further comprises an integrated circuit 202 connected to the back surface of substrate 102. The integrated circuit 202 is connected to the metal traces 118, 119, and 120, which enables additional miniaturization of the biosensor since electrical inputs and outputs can be programmed, controlled, and recorded by the integrated circuit 202. This embodiment is displayed in FIG. 16 from a different perspective. An orthogonal side view is displayed to show how target analytes 116 bind to high affinity species 115 on the surface of nanotube 103 during sensing and sample analysis.

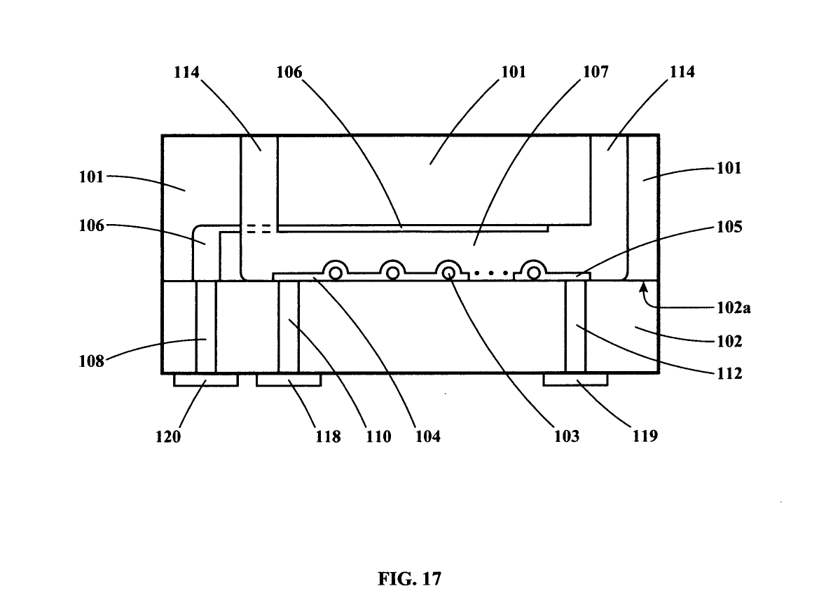

A slightly different embodiment is described with reference to FIG. 17. In this embodiment, an array of nanotubes 103 are horizontally grown or deposited on surface 102a, and microchannels 107 are etched or mechanically formed on the front surface of substrate 101. Gate electrode 106 runs along microchannel 107 and is connected to metal trace 120 via TSVs 108. Source electrode 104 on surface 102a is connected to TSV 110, which is connected to metal trace 118. Similarly, drain electrode 105 is connected to TSV 112, which is connected to metal trace 119. This integrates source electrode 104, nanotubes 103, gate electrode 106, and drain electrode 105 into one or a plurality of functional NT-FETs. Substrate 101 comprises microchannels 107, which are connected to vertical channels 114 to enable the introduction and exit of samples for sensing and analysis.

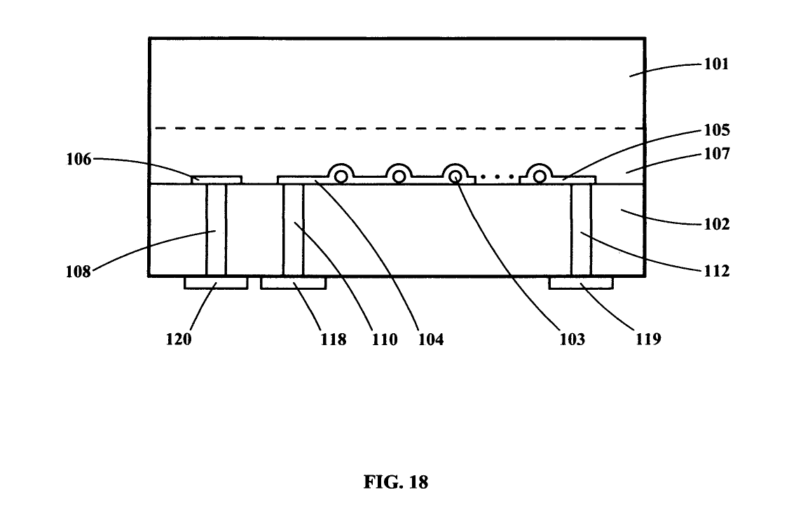

A different embodiment is described with reference to FIG. 18. In this embodiment, one or multiple nanotubes 103 are deposited or grown on substrate 102 and these are connected to source electrode 104 at one end and to drain electrode 105 at the second end. Source electrode 104 is electrically connected to metal trace 118 via TSV 110. Drain electrode 105 is electrically connected to metal trace 119 via TSV 112. Gate electrode 106 is located on the front surface of substrate 102, and it is electrically connected to metal trace 120 via TSV 108. Microchannel 107 is formed on the front surface of substrate 101, and said microchannel 107 runs along the width of the device as described in FIG. 18. Microchannel 107 provides for the introduction and exit of a sample during analysis.

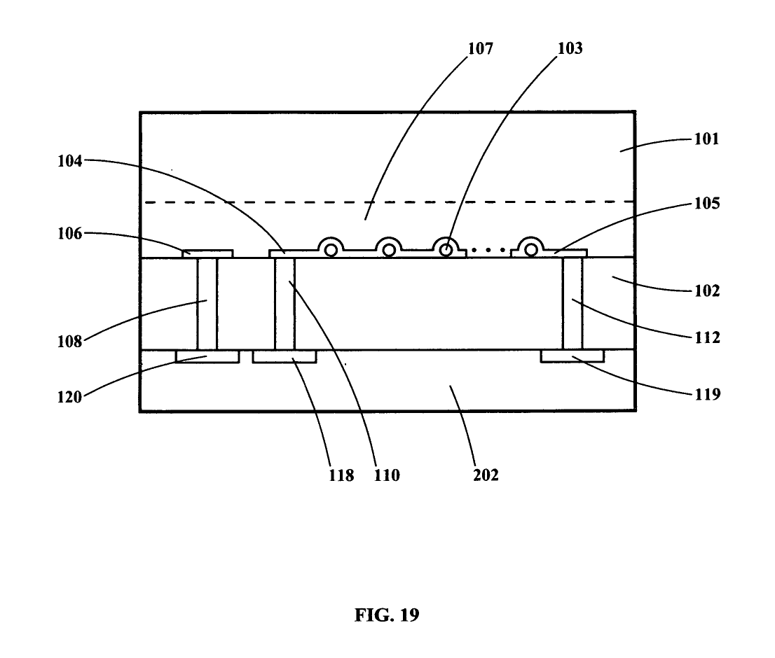

A slightly different embodiment is described with reference to FIG. 19. In addition to including all the elements described in FIG. 18, this embodiment comprises an integrated circuit 202 attached to the back surface of substrate 102. Said integrated circuit 202 is electrically connected to metal traces 118, 119, and 120, and can consequently control the electrical inputs and record electrical outputs of the NT-FET formed by the electrodes 104, 105, 106, and nanotubes 103.

Utility and Functional Advantages

Since the microfluidic and the electronic controls are decoupled to the opposite sides of the device as described in FIG. 4-8 and FIG. 10-19, other complementary operations can be added to the device. For instance, if substrates 101 and 102 are transparent, or translucent to light, and a light source (e.g., laser, UV, infrared, or visible) is illuminated from one side of the device, then fluorescence light and/or optical output can be collected and measured from the opposite side of the device for the case of the embodiments described in FIG. 4-6, FIG. 10-11, FIG. 13-14, and FIG. 17-18, which are embodiments that do not comprise an integrated circuit 202. If only one substrate is transparent or translucent, substrate 101 or 102, and the other substrate reflects light (e.g., laser, UV, IR or visible), then a light source and an output detector can be placed on the same side of the present invention. Consequently, fluorescence light and/or optical output can be collected and measured. These utility advantages are particularly relevant with respect to the embodiments described in FIG. 4-8 and FIG. 10-19. Using an external light source (e.g., laser, UV, IR or visible), the light is used to trigger photo-interactions between the high affinity species (e.g., nucleic acids, aptamers, antibodies, or antibody fragments) on the nanotubes with the analyte species of interest contained in the sample. These photo-interactions facilitate complementary forms of molecular characterization using optical means (e.g., laser, optical fluorescence, fluorescence resonance energy transfer (FRET), or other).

Molecular Detection, Sensing, and Analysis Method

A method according to the present invention is described in relation to FIG. 2-8, FIG. 10, FIG. 13, and FIG. 17. A solution of known concentration containing nucleic acids, antibodies, antibody fragments, enzymes, or engineered antibody fragments, or a combination thereof is introduced into sensing cavity 200 or microchannels 107 to coat, functionalize, and add target affinity to nanotubes 103. Nucleic acid molecules (e.g. aptamers), antibody molecules, antibody fragments, or engineered antibody fragments 115 bind to nanotubes 103.

A buffer electrolyte solution is introduced into the sensing cavity 200 or microchannels 107. The electrolyte solution permits the activation of the NT-FETs at their baseline current/voltage (I/V), which defines a reference state and it is equivalent to zero concentration of the measured targeted specie or analyte (e.g., protein biomarkers). This step is executed as part of the calibration procedure of the present invention. Subsequently, in order to complete the calibration, a solution containing high affinity and selectivity species (e.g., nucleic acids, aptamers, enzymes, antibodies, antibody fragments, engineered antibody fragments, or a combination thereof) and a reagent of known concentration are mixed with the buffer solution and introduced into the device to functionalize the surface of the nanotubes 103 with the high affinity and selectivity species 115.

Finally, the sample (e.g., known quantity of blood, plasma serum, or biological fluid) is mixed with known quantities of an electrolyte solution and/or reagents in order to be introduced into the sensing cavity 200 or the sensing microchannel 107. The arrays of NT-FETs at the bottom of the sensing cavity 200 or inside each sensing microchannel 107 serve as signal amplifiers and enable the recording of changes in I/V characteristics caused by the binding between the high affinity ligands 115 and the targeted analytes 116 (e.g., protein biomarkers) on the surface of the nanotubes 103. For example, in a blood serum analysis, the recorded I/V characteristics for a specific ligand-analyte pair 115-116 on the nanotubes 103 will be directly correlated to the concentration of said analyte 116 in the sample. The compilation of measurements of multiple types of analyte proteins 116 defines a signature-analyte-profile or signature-protein-profile (SAP), which is unique to each individual sample (e.g., blood serum sample).

Sensing cavity 200 or microchannels 107 may be cleaned and reused. This is done by flushing the sensing cavity 200 or microchannels 107 with a cleaning solution and re-functionalizing the nanotubes 103 with a new set of high affinity and selective species 115. A subsequent analysis with the same or different set of target analytes 116 (e.g., proteins) is performed to gather more information for the signature-analyte-profile (SAP).

### LIST OF ELEMENTS

The following is a list of elements comprised in the present invention.

| Number | Element |
|---|---|
| 101 | First layer or substrate |
| 101a | front surface of substrate 101 |
| 101b | back surface of substrate 101 |
| 102 | Second layer or substrate |
| 102a | front surface of substrate 102 |
| 102b | back surface of substrate 102 |
| 103 | Single-walled carbon nanotubes (SWCNT), also referred to as |
| | nanotube or nanotubes |
| 104 | source electrode |
| 105 | drain electrode |
| 106 | gate electrode |
| 107 | microchannel or microchannels, also referred to as sensing |
| | microchannel |
| 108 | gate TSV, where through-substrate via (TSV) |
| 110 | source TSV |
| 112 | drain TSV |
| 114 | vertical channel or channels |
| 115 | high affinity and selectivity species |
| 116 | target analytes |
| 117 | external gate electrode probe |
| 118 | source metal trace |
| 119 | drain metal trace |
| 120 | gate metal trace |
| 200 | through-substrate cavity (TSC), also referred to as sensing TSC |
| | cavity |
| 202 | integrated circuit |

1. A multilayer device for sensing metal ions, biological molecules, or whole cells, wherein the device comprises:
a) one or more cavities that provide for the introduction of a sample to be analyzed and one or more channels that provide for exit of the sample, or one or more channels that provide for the introduction and exit of the sample;
b) one or more single-walled carbon nanotubes presented to the one or more cavities or one or more channels;
c) a plurality of electrodes electrically connected to the one or more single-walled carbon nanotubes; and,
d) a reference gate electrode presented to the one or more cavities or one or more channels.

2. The device according to claim 1, wherein the one or more channels are at least 4 microns in width, at least 40 microns in length, and at least 3 microns in height.

3. The device according to claim 1, wherein the one or more single-walled carbon nanotubes are at least 2 microns long and positioned either in parallel or in series with one another or a combination thereof while being electrically connected to the electrodes.

4. The device according to claim 1, wherein the reference gate electrode is composed of a metal or a metallic alloy, and said gate electrode is located on a channel wall opposite or adjacent to that of the carbon nanotubes.

5. The device according to claim 1, wherein the reference gate electrode is presented to one or more cavities.

6. The device according to claim 1, wherein the reference gate electrode is presented to one or more channels.

7. The device according to claim 1, wherein the device further comprises a plurality of through layer conductive elements which provide short paths for electrical conduction.

8. The device according to claim 5, wherein the device comprises a first layer and a second layer, and wherein the first layer comprises a sensing cavity and microchannels allowing for the introduction and exit of the sample, and wherein the second layer comprises the one or more single-walled carbon nanotubes, a source electrode and a drain electrode, and wherein the reference gate electrode is an external gate electrode inserted into the sensing cavity.

9. The device according to claim 5, wherein the device comprises a first layer and a second layer, and wherein the first layer comprises a sensing cavity and microchannels allowing for the introduction and exit of the sample, and wherein the reference gate electrode runs along the sidewall of the sensing cavity and extends to the top of the first layer, and wherein the second layer comprises the one or more single-walled carbon nanotubes, a source electrode and a drain electrode.

10. The device according to claim 5, wherein the device comprises a first layer and a second layer, and wherein the first layer comprises a sensing cavity and microchannels allowing for the introduction and exit of the sample, and wherein the second layer comprises the one or more single-walled carbon nanotubes, a source electrode, a drain electrode and a plurality of through layer conductive elements, which provide short paths for electrical conduction, and wherein the source electrode is connected to a first through-layer conductive element and the drain electrode is connected to a second through-layer conductive element, and wherein the first conductive element is connected to a first metal trace on the external surface of the second layer, and wherein the second conductive element is connected to a second metal trace on the external surface of the second layer, and wherein the reference gate electrode is an external gate electrode inserted into the sensing cavity.

11. The device according to claim 5, wherein the device comprises a first layer and a second layer, and wherein the first layer comprises a sensing cavity and microchannels allowing for the introduction and exit of the sample, and wherein the second layer comprises the one or more single-walled carbon nanotubes, a source electrode, and a drain electrode, and wherein the source electrode is connected to a first through-layer conductive element and the drain electrode is connected to a second through-layer conductive element, and wherein the first conductive element is connected to a first metal trace on the external surface of the second layer, and wherein the second conductive element is connected to a second metal trace on the external surface of the second layer, and a third through-layer conductive element connecting the reference gate to a third metal trace on the external surface of the second layer.

12. The device according to claim 5, wherein the device comprises a first layer and a second layer, and wherein the first layer comprises a sensing cavity and microchannels allowing for the introduction and exit of the sample, and wherein the reference gate electrode runs along the sidewall of the sensing cavity and extends to the top of the first layer, and wherein the second layer comprises the one or more single-walled carbon nanotubes, a source electrode, and a drain electrode, and wherein the source electrode is connected to a first through-layer conductive element and the drain electrode is connected to a second through-layer conductive element, and wherein the first conductive element is connected to a first metal trace on the external surface of the second layer, and wherein the second conductive element is connected to a second metal trace on the external surface of the second layer.

13. The device according to claim 10, wherein the device further comprises a third layer, and wherein the third layer is an integrated circuit attached to the external surface of the second layer, and wherein the integrated circuit is connected to the first, second and third metal traces.

14. The device according to claim 11, wherein the device further comprises a third layer, and wherein the third layer is an integrated circuit attached to the external surface of the second layer, and wherein the integrated circuit is connected to the first, second and third metal traces.

15. The device according to claim 6, wherein the device comprises a first layer and a second layer, and wherein the first layer comprises vertical channels allowing for the introduction of the sample to a microchannel and exit of the sample from said microchannel, and the one or more single-walled carbon nanotubes on an internal surface of the first layer connected to a source electrode and a drain electrode, and wherein the second layer comprises the microchannel, a reference gate electrode, a first through-layer conductive element connecting the source electrode to a first metal trace on the external surface of the second layer, a second through-layer conductive element connecting the drain electrode to a second metal trace on the external surface of the second layer, and a third through-layer conductive element connecting the reference gate to a third metal trace on the external surface of the second layer.

16. The device according to claim 6, wherein the device comprises a first layer and a second layer, and wherein the first layer comprises vertical channels allowing for the introduction of the sample to a microchannel and exit of the sample from said microchannel, and the one or more single-walled carbon nanotubes on an internal surface of the first layer, and wherein the second layer comprises the microchannel, a reference gate electrode running along the microchannel on the internal surface of the second layer, a source electrode connected to a first through-layer conductive element that is further connected to a first metal trace on the external surface of the second layer, a drain electrode connected to a second through-layer conductive element that is further connected to a second metal trace on the external surface of the second layer, wherein the reference gate electrode is connected to a third through-layer conductive element that is further connected to a third metal trace on the external surface of the second layer.

17. The device according to claim 6, wherein the device comprises a first layer and a second layer, and wherein the first layer comprises vertical channels allowing for the introduction of the sample to a microchannel and exit of the sample from said microchannel, and the microchannel, and wherein the second layer comprises the one or more single-walled carbon nanotubes on the internal surface of the second layer, a source electrode connected to a first through-layer conductive element that is further connected to a first metal trace on the external surface of the second layer, a drain electrode connected to a second through-layer conductive element that is further connected to a second metal trace on the external surface of the second layer and a reference gate electrode connected to a third through-layer conductive element that is further connected to a third metal trace on the external surface of the second layer.

18. The device according to claim 6, wherein the device comprises a first layer and a second layer, and wherein the first layer comprises vertical channels allowing for the introduction of the sample to a microchannel and exit of the sample from said microchannel, the microchannel and a reference gate electrode running along the microchannel, and wherein the second layer comprises a first through-layer conductive element that connects the reference gate electrode and a first metal trace on the external surface of the second layer, the one or more single-walled carbon nanotubes on the internal surface of the second layer, a source electrode connected to a second through-layer conductive element that is further connected to a second metal trace on the external surface of the second layer, and a drain electrode connected to a third through-layer conductive element that is further connected to a third metal trace on the external surface of the second layer.

19. The device according to claim 6, wherein the device comprises a first layer and a second layer, and wherein the first layer comprises a microchannel running along the internal surface of the first layer that allows for the introduction and exit of the sample, and wherein the second layer comprises the one or more single-walled nanotubes on the internal surface of the second layer, a source electrode connected to a first through-layer conductive element that is further connected to a first metal trace on the external surface of the second layer, a drain electrode connected to a second through-layer conductive element that is further connected to a second metal trace on the external surface of the second layer, and a reference gate electrode connected to a third through-layer conductive element that is further connected to a third metal trace on the external surface of the second layer.

20. The device according to claim 15, wherein the device further comprises a third layer, and wherein the third layer is an integrated circuit attached to the external surface of the second layer, and wherein the integrated circuit is connected to the first, second and third metal traces.

21. The device according to claim 17, wherein the device further comprises a third layer, and wherein the third layer is an integrated circuit attached to the external surface of the second layer, and wherein the integrated circuit is connected to the first, second and third metal traces.

22. The device according to claim 19, wherein the device further comprises a third layer, and wherein the third layer is an integrated circuit attached to the external surface of the second layer, and wherein the integrated circuit is connected to the first, second and third metal traces.

23. A method for sensing species such as a metal, biological cells, and one or more biological molecules, wherein the method comprises the steps of:
a) introducing a solution of high affinity and selective binding elements into a device according to claim 1, wherein the high affinity and selective binding elements add functionality to the one or more single-walled carbon nanotubes by binding species of interest to the surface of the nanotubes;
b) introducing a buffer-electrolyte solution into one or more cavities or one or more channels of the device, thereby allowing activation of nanotube-field effect transistors in the device for calibration and for setting a baseline current or voltage reference state;
c) introducing a sample in solution with a buffer-electrolyte solution into the one or more channels of the device and determining any changes in the current or voltage state of the nanotube-field effect transistors relative to their baseline state;
wherein the changes are correlated with the binding of one or more species of interest in the sample.

24. The method according to claim 23, wherein high affinity and selectivity binding elements are selected from a group of elements consisting of nucleic or oligonucleic acid molecules, peptides, enzymes, monoclonal antibodies, polyclonal antibodies, minibodies, diabodies, cys-diabodies, derived antibody fragments and fab fragments.

25. The method according to claim 23, wherein the buffer-electrolyte solution promotes ionic exchange and transport, and wherein the pH ranges from 4.0 to 10.0.
